(19) United States
(12) Patent Application Publication
(10) Pub. No.: US 2014/0335052 A1
(43) Pub. Date: Nov. 13, 2014

(54) COMBINATION THERAPY FOR TREATING HCV INFECTION IN AN HCV-HIV COINFECTED PATIENT POPULATION

(71) Applicant: Jens J. KORT, Danbury, CT (US)

(72) Inventor: Jens J. KORT, Danbury, CT (US)

(73) Assignee: BOEHRINGER INGELHEIM INTERNATIONAL GMBH, Ingelheim am Rhein (DE)

(21) Appl. No.: 14/446,384

(22) Filed: Jul. 30, 2014

Related U.S. Application Data
(63) Continuation of application No. 13/778,725, filed on Feb. 27, 2013.
Application No. 13/778,725 is a continuation of application No. PCT/US2012/029011, filed on Mar. 14, 2012.

(51) Int. Cl. A61K 38/21; A61K 31/7056; A61K 31/4709

(52) U.S. Cl. CPC A61K 38/212; A61K 31/4709; A61K 31/7056
USPC 424/85.7

(57) ABSTRACT

The present invention relates to therapeutic combinations comprising (a) Compound (1), or a pharmaceutically acceptable salt thereof, as herein described, (b) an interferon alfa and (c) ribavirin. Compound (1) is a selective and potent inhibitor of the HCV NS3 serine protease. The present invention also relates to methods of using such therapeutic combinations for treating HCV infection or alleviating one or more symptoms thereof in a patient that is co-infected with HIV.

### TECHNICAL FIELD OF THE INVENTION

The present invention relates to therapeutic combinations comprising Compound (1) as herein described, an interferon alfa and ribavirin. The present invention also relates to methods of using such therapeutic combinations for treating HCV infection or alleviating one or more symptoms thereof in a patient that is co-infected with the Human Immunodeficiency Virus (HIV). The present invention also provides kits comprising the therapeutic combinations of the present invention.

### BACKGROUND OF THE INVENTION

The following Compound (1):

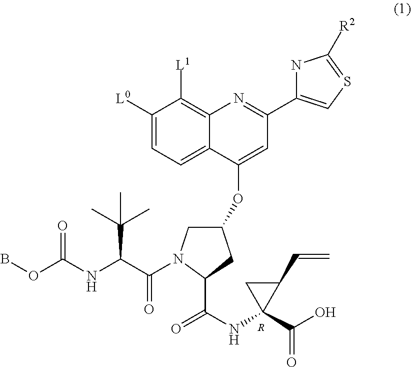

wherein B is

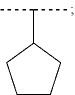

### L⁰ is MeO—; L¹ is Br; and R² is

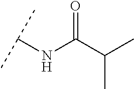

and having the chemical name: 1-{[4-[8-Bromo-2-(2-isopropylcarbamoyl-thiazol-4-yl)-7-methoxy-quinolin-4-yloxy]-1-(R)-(2-cyclopentyloxycarbonyl amino-3,3-(S)-dimethyl-butyryl)-pyrrolidine-(S)-2-carbonyl]-amino}-2-(S)-vinyl-cyclopropane-(R)-carboxylic acid, is known as a selective and potent inhibitor of the HCV NS3 serine protease and useful in the treatment of HCV infection. Compound (1) falls within the scope of the acyclic peptide series of HCV inhibitors disclosed in U.S. Pat. Nos. RE 40,525, 7,514,557 and 7,585,845. Compound (1) is disclosed specifically as Compound #1055 in U.S. Pat. No. 7,585,845, and as Compound #1008 in U.S. Pat. No. 7,514,557. Compound (1), and pharmaceutical formulations thereof, can be prepared according to the general procedures found in the above-cited references, all of which are herein incorporated by reference in their entirety. Preferred forms of Compound (1) include the crystalline forms, in particular the crystalline sodium salt form, which can be prepared as described in U.S. Patent Application Publication No. 2010/0093792, also incorporated herein by reference.

Data demonstrating the activity of Compound (1) as an inhibitor of the HCV NS3 serine protease and its corresponding demonstrated utility in the treatment of HCV infection in mono-infected patients, can be found in U.S. Pat. No. 7,585,845, as well as in numerous publications presenting the preclinical characterization or clinical trial results with Compound (1). See, e.g., Sulkowski M S, et al, Hepatol (2009), Vol. 50, pg. 2A, Abstract LB3; Sulkowski M S, et al., J Hepatol (2010) Vol. 52, Supp. 1, pgs. S462-S463, Abstract 1190; Berg et al., Hepatol (2010), Vol. 52, Supp. S1, Abstract 804; and White P W, et al., Antimicrob Agents Chemother (2010) 54(11): 4611-4618.

Combination therapy regimens directed to administering Compound (1) with an interferon-alpha and ribavirin for the treatment of HCV infection are described in U.S. Patent Application Publication Nos. 2010/0068182 and 2011/0268700.

HIV/HCV coinfected persons tend to have higher HCV viral loads and are less likely to clear the HCV spontaneously. The urgency for treatment of persons who are coinfected is greater than it is for those with HCV infection alone. The course of liver disease is more rapid in HIV/HCV-coinfected persons, including an approximately 2-fold increased risk of cirrhosis, more rapid progression to decompensated liver disease and increased risk for hepatocellular carcinoma (Graham C S, et al., Clin Infect Dis (2001); 33:562-569) Treatment of HCV might improve the tolerability of highly active antiretroviral therapy (HAART) because HCV infection increases the risk of mitochondrial toxicity and hepatotoxicity from HAART (Sulkowski M S, et al., JAMA (2000); 283:74-80; Lafeuillade A, et al., Lancet (2001); 357:280-281). Although there is much less published information on treatment outcomes in those who are HIV/HCV-coinfected than in HCV mono-infected patients, all accumulated data demonstrate that sustained virological response (SVR) and cure from HCV infection with pegylated interferon alpha and ribavirin is achieved in a substantially lower proportion of HIV/HCV coinfected patients when compared to HCV mono-infected patients. Factors associated with a poor treatment response (e.g., a high baseline HCV viral load, cirrhosis, and African American race) are present in a higher proportion of HIV/HCV coinfected populations, when compared to HCV monoinfected populations. It is not clear to what extent HIV infection itself diminishes the SVR rate, and to what extent advanced immunosuppression (e.g., CD4⁺ T lymphocyte count <200/mm³) further reduces response to HCV treatment (Toriani F J, et al., N Engl J Med (2004); 351(5): 438-50; Nunez M, et al., ARHR (2007); 23(8):972-982).

Thus, there is a continuing high unmet need in the art for therapies that are effective against HCV in patients that are co-infected with HIV.

### BRIEF SUMMARY OF THE INVENTION

In view of the known potency of Compound (1) as an inhibitor of the HCV NS3 serine protease and its corresponding demonstrated utility in the treatment of HCV infection in mono-infected patients, Applicants have tested this compound in HCV patients that are co-infected with HIV. Surprisingly, the results have demonstrated that the overall efficacy profile for this treatment regimen in HCV/HIV co-infected patients is quite good and even comparable to that seen in HCV monoinfected patients. Such results are surprising given that the HCV/HIV co-infected patient population is traditionally a much more difficult-to-treat patient population that is less responsive to traditional anti-HCV therapy than is the HCV mono-infected population.

The present invention provides a method of treating HCV infection or alleviating one or more symptoms thereof in a patient that is co-infected with HIV comprising the step of administering to the patient a therapeutic combination comprising a Compound (1) as defined herein, or a pharmaceutically acceptable salt thereof, together with an interferon alfa and ribavirin, as defined herein. The three actives of the combination can be administered simultaneously or separately, as part of a regimen.

The present invention further provides for a packaged pharmaceutical composition comprising a packaging containing one or more doses of Compound (1), or a pharmaceutically acceptable salt thereof and (b) written instructions directing the co-administration of Compound (1), or a pharmaceutically acceptable salt thereof, interferon alpha, and ribavirin for the treatment of HCV infection in a patient that is co-infected with HIV.

### DETAILED DESCRIPTION OF THE INVENTION

### Definitions

“Compound (1)” is as defined above.

“HCV infection” as used herein means infection by any subtype of the Hepatitis C Virus, including subtypes 1-6, and includes both acute and chronic HCV infection.

“Interferon” means a member of a family of highly homologous species-specific proteins that inhibit viral replication and cellular proliferation and modulate immune response. Human interferons are grouped into three classes based on their cellular origin and antigenicity: α-interferon (leukocytes), β-interferon (fibroblasts) and γ-interferon (B cells), Recombinant forms of each group have been developed and are commercially available. Subtypes in each group are based on antigenic/structural characteristics. At least 24 interferon alfas (grouped into subtypes A through H) having distinct amino acid sequences have been identified by isolating and sequencing DNA encoding these peptides. The terms “α-interferon”, “alfa-interferon” and “interferon alfa” are used interchangeably in this application to describe members of this group. Both naturally occurring and recombinant alfa-interferons, including consensus interferon, may be used in the practice of the invention.

Suitable interferon-alfas for the present invention include, but are not limited to, recombinant interferon alfa-2b such as INTRON®-A interferon and VIRAFERON®; recombinant interferon alfa-2a such as ROFERON® interferon; recombinant interferon alfa-2c such as BEROFOR® alfa 2 interferon; interferon alfa-n1, a purified blend of natural alfa interferons such as SUMIFERON® or WELLFERON® interferon alfa-n1 (INS); or a consensus alfa interferon such as those described in U.S. Pat. Nos. 4,897,471 and 4,695,623; or interferon alfa-n3, a mixture of natural alfa interferons such as ALFERON®. The use of interferon alfa-2a or alfa 2b is preferred. The manufacture of interferon alfa 2b is described in U.S. Pat. No. 4,530,901.

The term “interferon alfa” is further intended to include those “pegylated” analogs meaning polyethylene glycol modified conjugates of interferon alfa, preferably interferon alfa-2a and -2b. The preferred polyethylene-glycol-interferon alfa-2b conjugate is PEG₁₂₀₀₀-interferon alfa 2b. The term “PEG₁₂₀₀₀-IFN alfa” as used herein means conjugates such as are prepared according to the methods of International Application No. WO 95/13090 and containing urethane linkages between the interferon alfa-2a or -2b amino groups and polyethylene glycol having an average molecular weight of 12000.

The preferred PEG₁₂₀₀₀-interferon alfa-2b is prepared by attaching a PEG polymer to the epsilon amino group of a lysine residue in the IFN alfa-2b molecule. A single PEG₁₂₀₀₀ molecule is conjugated to free amino groups on an IFN alfa-2b molecule via a urethane linkage. This conjugate is characterized by the molecular weight of PEG₁₂₀₀₀ attached. The PEG₁₂₀₀₀-IFN alfa-2b conjugate is formulated as a lyophilized powder for injection. The objective of conjugation of IFN alfa with PEG is to improve the delivery of the protein by significantly prolonging its plasma half-life, and thereby provide protracted activity of IFN alfa.

Especially preferred conjugates of interferon alfa that may be used in the present invention are pegylated alfa-interferons, e.g., pegylated interferon alfa-2a, pegylated interferon alfa-2b, pegylated consensus interferon or pegylated purified interferon alfa product. Pegylated interferon alfa-2a is described, e.g., in European Patent No. EP 0 593 868 and commercially-available, e.g., under the tradename PEGASYS® (Hoffmann-La Roche). Pegylated interferon alfa-2b is described, e.g., in U.S. Pat. No. 5,908,621 and WO 98/48840 and commercially-available, e.g., under the tradename PEG-INTRON® A (Schering Plough). Pegylated consensus interferon is described in WO 96/11953. The preferred pegylated alfa interferons are pegylated interferon alfa-2a and pegylated interferon alfa-2b. Also preferred is pegylated consensus interferon.

The term “interferon alfa” further includes other interferon alfa conjugates that can be prepared by coupling an interferon alfa to a water-soluble polymer. A non-limiting list of such polymers includes other polyalkylene oxide homopolymers such as polyethylene glycol (PEG), polypropylene glycols, polyoxyethylenated polyols, copolymers thereof and block copolymers thereof. As an alternative to polyalkylene oxide-based polymers, effectively non-antigenic materials such as dextran, polyvinylpyrrolidones, polyacrylamides, polyvinyl alcohols, carbohydrate-based polymers and the like can be used. Such interferon alfa-polymer conjugates are described in U.S. Pat. No. 4,766,106, U.S. Pat. No. 4,917,888, European Patent Application No. 0 236 987, European Patent Application Nos. 0510 356, 0 593 868 and 0 809 996 (pegylated interferon alfa-2a) and International Publication No. WO 95/13090.

The term “interferon alfa” further includes fusion proteins of an interferon alfa, for example fusion proteins of interferon-α-2a, interferon-α-2b, consensus interferon or purified interferon-α product, each of which is fused with another protein. Certain preferred fusion proteins comprise an interferon (e.g., interferon-α-2b) and an albumin as described in U.S. Pat. No. 6,972,322 and international publications WO2005/003296 and WO2005/077042. Also included are consensus interferons, such as INFERGEN®.

The term “pharmaceutically acceptable salt” means a salt of a Compound of formula (1) which is, within the scope of sound medical judgment, suitable for use in contact with the tissues of humans and lower animals without undue toxicity, irritation, allergic response, and the like, commensurate with a reasonable benefit/risk ratio, generally water or oil-soluble or dispersible, and effective for their intended use.

The term includes pharmaceutically-acceptable acid addition salts and pharmaceutically-acceptable base addition salts. Lists of suitable salts are found in, e.g., S. M. Birge et al., J. Pharm. Sci., 1977, 66, pp. 1-19.

The term “pharmaceutically-acceptable acid addition salt” means those salts which retain the biological effectiveness and properties of the free bases and which are not biologically or otherwise undesirable, formed with inorganic acids such as hydrochloric acid, hydrobromic acid, sulfuric acid, sulfamic acid, nitric acid, phosphoric acid, and the like, and organic acids such as acetic acid, trifluoroacetic acid, adipic acid, ascorbic acid, aspartic acid, benzenesulfonic acid, benzoic acid, butyric acid, camphoric acid, camphorsulfonic acid, cinnamic acid, citric acid, digluconic acid, ethanesulfonic acid, glutamic acid, glycolic acid, glycerophosphoric acid, hemisulfic acid, hexanoic acid, formic acid, fumaric acid, 2-hydroxyethane-sulfonic acid (isethionic acid), lactic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, pivalic acid, propionic acid, pyruvic acid, salicylic acid, stearic acid, succinic acid, sulfanilic acid, tartaric acid, p-toluenesulfonic acid, undecanoic acid, and the like.

The term “pharmaceutically-acceptable base addition salt” means those salts which retain the biological effectiveness and properties of the free acids and which are not biologically or otherwise undesirable, formed with inorganic bases such as ammonia or hydroxide, carbonate, or bicarbonate of ammonium or a metal cation such as sodium, potassium, lithium, calcium, magnesium, iron, zinc, copper, manganese, aluminum, and the like. Particularly preferred are the ammonium, potassium, sodium, calcium, and magnesium salts. Salts derived from pharmaceutically-accepta-ble organic nontoxic bases include salts of primary, secondary, and tertiary amines, quaternary amine compounds, substituted amines including naturally occurring substituted amines, cyclic amines and basic ion-exchange resins, such as methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, methylglucamine, theobromine, purines, piperazine, piperidine, N-ethylpiperidine, tetramethylammonium compounds, tetraethylammonium compounds, pyridine, N,N-dimethylaniline, N-methylpiperidine, N-methylmorpholine, dicyclohexylamine, dibenzylamine, N,N-dibenzylphenethylamine, 1-ephenamine, N,N′-dibenzylethylenediamine, polyamine resins, and the like. Particularly preferred organic nontoxic bases are isopropylamine, diethylamine, ethanolamine, trimethylamine, dicyclohexylamine, choline, and caffeine.

“Ribavirin” refers to 1-β-D-ribofuranosyl-1H-1,2,4-triazole-3-carboxamide, available from ICN Pharmaceuticals, Inc., Costa Mesa, Calif. and is described in the Merck Index, compound No. 8199, Eleventh Edition. Its manufacture and formulation is described in U.S. Pat. No. 4,211,771. Preferred marketed ribavirin products include REBETOL® and COPEGUS®. The term further includes derivatives or analogs thereof, such as those described in U.S. Pat. Nos. 6,063,772, 6,403,564 and 6,277,830. For example, derivatives or analogs include modified ribavirins such as 5′-amino esters, ICN Pharmaceutical's L-enantiomer of ribavirin (ICN 17261), 2′-deoxy derivatives of ribavirin and 3-carboxamidine derivatives of ribavirin, viramidine (previously known as ribamidine) and the like.

The term “therapeutic combination” as used herein means a combination of one or more active drug substances, i.e., compounds having a therapeutic utility. Typically, each such compound in the therapeutic combinations of the present invention will be present in a pharmaceutical composition comprising that compound and a pharmaceutically acceptable carrier. The compounds in a therapeutic combination of the present invention may be administered simultaneously or separately, as part of a regimen.

### EMBODIMENTS OF THE INVENTION

According to a general embodiment, the present invention provides for a method of treating HCV infection or alleviating one or more symptoms thereof in a patient in a that is co-infected with HIV comprising the step of administering to the patient a therapeutic combination comprising a Compound (1) as defined herein, or a pharmaceutically acceptable salt thereof, together with an interferon alfa and ribavirin. In another embodiment, the present invention teaches the use of a Compound (1) as defined herein, or a pharmaceutically acceptable salt thereof, an interferon alfa, and ribavirin for the preparation of a pharmaceutical kit to treat a hepatitis C viral (HCV) infection or alleviating one or more symptoms thereof in a patient in a patient that is co-infected with HIV.

In administering the therapeutic combinations of the present invention, each active agent can be administered together at the same time or separately at different times in separate dosage administrations. The present invention contemplates and includes all such dosage regimens when administering the triple therapeutic combinations as defined herein.

Although this combination therapy is expected to be effective against all HCV genotypes, it has been demonstrated to be particularly effective in treating HCV genotype 1 infection.

A preferred embodiment is directed to the treatment of patients have the HCV subtype 1 a which represent particularly difficult-to-treat HCV-infected patient populations. The patient population to be treated with the combination therapy of the present invention can be further classified into “treatment-naïve” patients, i.e., those patient who have not received any prior treatment for HCV infection and “treatment experienced” patients, i.e, those patients who have undergone prior treatment for HCV. Either of these classes of patients may be treated with the combination therapy of the present invention. A particular class of patients that are preferably treated are those treatment experienced patients that have undergone prior interferon plus ribavirin therapy but are non-responsive to said therapy (herein “non-responders”). Such non-responders include three distinct groups of patients: (1) those who experienced <1×log₁₀ maximum reduction in HCV RNA levels during treatment with interferon plus ribavirin (“null responders”), (2) those who experienced ≧1×log₁₀ maximum reduction in HCV RNA levels during treatment with interferon plus ribavirin but never achieve HCV RNA levels below level of detection (“partial responders”), and (3) those who achieved a virologic response with and during interferon plus ribavirin therapy but had a viral load rebound after treatment has completed (“relapser”). Another treatment experienced patient population to be treated with the combination therapy of the present invention includes those who achieved an initial virologic response with (pegylated) interferon plus ribavirin but had viral load rebound during treatment other than due to nonadherence to the treatment.

According to an alternative embodiment, the present invention provides a method of reducing HCV-RNA levels in a patient in need thereof, comprising the step of administering to said patient a therapeutic combination according to the present invention. Preferably, the method of the present invention reduces the HCV-RNA levels in a patient to a level below the lower limit of quantification (or “BLQ”). A BLQ level of HCV RNA as used in the present invention means a level below 25 International Units (IU) per ml of serum or plasma of a patient as measured by quantitative, multi-cycle reverse transcriptase PCR methodology according to the WHO international standard (Saladanha J, Lelie N and Heath A, Establishment of the first international standard for nucleic acid amplification technology (NAT) assays for HCV RNA. WHO Collaborative Study Group. Vox Sang 76:149-158, 1999). Such methods are well known in the art. In preferred embodiments, the method of the present invention reduces the HCV-RNA levels in a patient to less than 25 IU per ml of serum or plasma, or less than 10 IU per ml of serum or plasma.

In another embodiment the method of the present invention reduces the HCV-RNA levels in a patient to less than a detectible level (below the limit of detection, BLD). Treatment decisions for duration of HCV therapy can be made based on BLD, and combinations of BLQ and BLD HCV RNA at subsequent timepoints during initial treatment. Typical time points include HCV RNA measurements at 4, 8, and 12 weeks after initiation of therapy, and results are utilized to guide further treatment duration “response-guided therapy”. Cure from HCV infection is typically inferred if HCV RNA remained BLD 12-24 weeks after end of HCV treatment. Thus, in additional embodiments, the method of the present invention results in an HCV-RNA level in the patient that is less than a detectible level at 12 weeks, preferably 24 weeks, after the end of all treatment.

The usual duration of the treatment for standard pegylated interferon plus ribavirin therapy in HIV/HCV coinfected patients is at least 48 weeks, and up to 72 weeks for chronic HCV infection with HCV genotype 1 or 4; 48 weeks for the majority of HIV/HCV coinfected patients with chronic HCV genotype 2 or 3 infection. A few patients with chronic HCV genotype 2 and 3 infection may be treated with 24 weeks of pegylated interferon alpha and ribavirin. However, with the addition of Compound (1), or a pharmaceutically acceptable salt thereof, in the triple combination therapy of the present invention, it may be possible to have a much shorter duration of treatment. With the triple combination therapy of the present invention the contemplated durations of treatment include at least 4 weeks, preferably at least 12 weeks, e.g., from about 12 weeks to about 24 weeks, although treatment up to and even beyond 48 weeks is possible as well. Thus, further embodiments include treatment for at least 24 weeks and for at least 48 weeks. The duration of treatment of chronic HCV infection may vary depending upon the specific HCV genotype. For example, the typical duration of treatment will be longer for genotypes 1 and 4, than for genotypes 2 and 3. In addition, the treatment duration will be shorter for the treatment of acute infection as compared to chronic infection. Also contemplated is an initial treatment regimen with the triple combination therapy of the present invention, followed by a continuation of only the interferon plus ribavirin double combination therapy. Thus, possible scenarios for the initial triple and then double combination therapy include, for example: (1) 4 weeks of the triple combination therapy, followed by 8 to 44 weeks of the interferon plus ribavirin only therapy; (2) 12 weeks of the triple combination therapy, followed by 0 to 36 weeks of the interferon plus ribavirin only therapy; and (3) 24 weeks of the triple combination therapy, followed by 0 to 24 weeks of the interferon plus ribavirin only therapy.

The patient to be treated with the combination therapy of the present invention is a patient that is co-infected with the Human Immunodeficiency Virus (HIV), including HIV-1 or HIV-2 infection. In a specific embodiment the HIV infection is chronic HIV-1 infection, as documented by seroconversion and presence of detectable anti-HIV antibodies. Patients that can be treated include those that are anti-retroviral naïve or on stable anti-retroviral therapy.

The patient is diagnosed as having chronic HIV infection using the standard diagnostic testing methods known in the art, e.g. HIV-1/HIV-2 antibody ELISA for screening, HIV Western blot for confirmation of a positive screening result, and quantitative assay for detection of HIV-1 or HIV-2 plasma RNA, which may detect HIV RNA early after HIV infection and before anti-HIV antibodies have developed.

Patients with HIV infection may be asymptomatic or classified as having advanced HIV disease=aquired immunodeficiency syndrome (AIDS) and regardless of peripheral absolute CD4⁺ T lymphocyte counts. However, patients with active AIDS defining illnesses as defined by CDC (Centers for Disease Control and Prevention, 1992 MMWR, 41(RR17), should not undergo triple therapy for HCV until AIDS defining illness has been sufficiently treated and has resolved.

In a specific embodiment of the invention, the HCV-infected patient to be treated is first tested to confirm the presence of an HIV co-infection in the patient. Thus, one embodiment is directed to a method of treating HCV infection or alleviating one or more symptoms thereof in a patient in comprising the step of administering to the patient a therapeutic combination comprising a Compound (1) as defined herein, or a pharmaceutically acceptable salt thereof, together with an interferon alfa and ribavirin, wherein the patient has first been identified as having an HIV co-infection. Various test methods for making this identification are known in the art, as discussed above.

The first component of the therapeutic combination, namely, Compound (1) or a pharmaceutically acceptable salt thereof is comprised in a composition. Such a composition comprises Compound (1), or a pharmaceutically acceptable salt thereof, and a pharmaceutically acceptable adjuvant or carrier. Typical pharmaceutical compositions that may be used for Compound (1), or a pharmaceutically acceptable salt thereof, are as described in U.S. Pat. No. 7,585,845, WO 2010/059667 and WO 2011/005646.

In general, the Compound (1) or a pharmaceutically acceptable salt thereof may be administered at a maintenance dosage of at least 40 mg/day (in single or divided doses). Additional embodiments for dosage amounts and ranges may include (in single or divided doses):
(a) at least 120 mg/day
(b) at least 240 mg/day
(c) at least 360 mg/day
(d) at least 480 mg/day
(e) from about 40 mg/day to about 480 mg/day
(f) from about 120 mg/day to about 240 mg/day
(g) from about 240 mg/day to about 480 mg/day
(h) about 120 mg/day
(i) about 240 mg/day
(j) about 360 mg/day
(k) about 480 mg/day

Although Compound (1) or a pharmaceutically acceptable salt thereof may be administered in single or divided daily doses, once a day administration (QD) of the daily dose is preferred. As the skilled artisan will appreciate, however, lower or higher doses than those recited above may be required. Specific dosage and treatment regimens for any particular patient will depend upon a variety of factors, including the age, body weight, general health status, sex, diet, time of administration, rate of excretion, drug concomitant medications (co-medications), the severity and course of the infection, the patient's disposition to the infection and the judgment of the treating physician. Specific factors affecting dosing may include, for example, individual patient factors which modify the adsorption, distribution, metabolism and excretion of Compound (1); the specific HCV Genotype; the specific IL28B genotype of the patient; the patient's innate/adaptive immune response to HCV; acute vs. chronic HCV infection; and the disposition of ribavirin based on host factors. In general, the compound is most desirably administered at a concentration level that will generally afford antivirally effective results without causing any harmful or deleterious side effects.

Typical dosing for HIV/HCV coinfected adults will be 120 mg QD and 240 mg QD. In the presence of a strong enzyme-inducer comedication, dosing higher than 240 mg QD may be possible. In the presence of a strong CYP3A inhibitor such as ritonavir or cobicistat as part of HIV therapy, a daily dose of less than 120 mg QD, for example in certain patients, may be possible (40 or 80 mg). Dosing for children starting at ages 3-5 may be substantially lower than 40 mg QD.

In another embodiment according to the invention, a loading dose amount of Compound (1) is administered for the first administration dose of the treatment. The loading dose amount is higher than the dose amount administered for subsequent administrations in the treatment, which are referred to as maintenance doses. Preferably, the loading dose amount is about double in quantity, by weight, of the amount in subsequent administrations in the treatment. For example, in one embodiment, the first dose of Compound (1) administered at loading dosage of about 240 mg and subsequent maintenance doses of Compound (1) are administered at a daily dosage of about 120 mg. In another embodiment, the first dose of Compound (1) administered at a loading dosage of about 480 mg and subsequent maintenance doses of Compound (1) are administered at a daily dosage of about 240 mg. By using this loading dose concept, a clear advantage is that it is thereby possible to achieve steady state levels of active drug in the patient's system earlier than would otherwise be achieved. A higher blood level is achieved early by using a loading dose, preferably double the maintenance dose at first intake. Reaching the targeted steady state level of active drug earlier in therapy also means that there is less possibility of insufficient drug exposure at the beginning of therapy so that resistant viral strains have a smaller chance of emerging.

The second component of the therapeutic combination, namely interferon-alfa, is comprised in a pharmaceutical composition. Typically, such compositions are injectible formulations comprising interferon-alfa and a pharmaceutically acceptable adjuvant or carrier and are well known in the art, including in a number of marketed interferon-alfa formulations. See, e.g., the various marketed interferon-alfa products and various patent and other literature related to interferon-alfa cited hereinabove.

The types of interferon-alfas that may be used in the combination are as outlined hereinabove in the definitions section. In one preferred embodiment, the interferon alfa is a pegylated interferon alfa. In a further embodiment, the interferon alfa is a pegylated interferon alfa-2a or pegylated interferon alfa-2b. In a particularly preferred embodiment, the interferon alfa is PEGASYS® or PEG-INTRON®.

When using known, marketed interferon alfa products, such products may be administered at their labeled dosage levels indicated for interferon plus ribavirin combination therapy for the treatment of HCV infection.

In one embodiment, the interferon alfa may be administered parenterally one to three times per week, preferably once or twice a week. With respect to pegylated interferon alfas, these are typically administered once per week and the total weekly dose ranges, e.g., from about 0.5 μg/kg/week to about 2 μg/kg/week in case of pegylated interferon alfa-2b, and with respect to pegylated interferon alfa-2a the dosage is independent from the body weight of the host and is typically about 90 to 180 μg/week, more preferably about 135 to about 180 μg/week. In combination with ribavirin, a standard dosage of pegylated interferon alfa-2b is about 1.5 μg/kg/week and a standard dosage of pegylated interferon alfa-2a is about 180 μg/week, together with ribavirin, which is preferably dosed once or twice daily according to body weight and with a total daily dose of about 200 to 1800 mg/day, in particular, 800-1200 mg/day of oral ribavirin.

According to further embodiments, the pegylated interferon alfa-2b may be administered at dosages of:
(a) about 0.5 μg/kg/week to about 2 μg/kg/week;
(b) about 1 μg/kg/week to about 2 μg/kg/week;
(c) about 1.5 μg/kg/week to about 2 μg/kg/week;
(d) about 1.5 μg/kg/week

According to further embodiments, the pegylated interferon alfa-2a may be administered at dosages of:
(a) about 90 to about 180 μg/week;
(b) about 135 to about 180 μg/week;
(b) about 90 μg/week

The third component of the therapeutic combination, namely ribavirin, is comprised in a pharmaceutical composition. Typically, such compositions comprise ribavirin and a pharmaceutically acceptable adjuvant or carrier and are well known in the art, including in a number of marketed ribavirin formulations. Formulations comprising ribavirin are also disclosed, e.g., in U.S. Pat. No. 4,211,771.

The types of ribavirin that may be used in the combination are as outlined hereinabove in the definitions section. In one preferred embodiment, the ribavirin is either REBETOL® or COPEGUS® and they may be administered at their labeled dosage levels indicated for interferon plus ribavirin combination therapy for the treatment of HCV infection. Of course, with the triple combination therapy of the present invention it may be possible to use a lower dosage of ribavirin, e.g., lower than is used the current standard interferon plus ribavirin therapy, while delivering the same or better efficacy than the current standard therapy with less side-effects usually associated with such therapy.

According to various embodiments, the ribavirin may be administered at dosages of (in single or divided doses):
(a) between 200 mg/day to about 1800 mg/day;
(b) between about 800 mg/day to about 1200 mg/day;
(c) between about 1000 mg/day to about 1200 mg/day;
(d) about 1000 mg/day
(e) about 1200 mg/day
(f) alternating doses 400 mg/day and 200 mg/day every other day
(g) between 400 mg/day to about 600 mg/day
(h) about 400 mg/day
(i) about 600 mg/day
(j) about 800 mg/day

According to one embodiment, the ribavirin composition comprises ribavirin in a formulation suitable for dosing once a day or twice daily. For example, if a therapeutic combination comprises about 1000 mg/day dosage of ribavirin, and a dosing of two times a day is desired, then the therapeutic combination will comprise ribavirin in a formulation, e.g., a tablet, containing, e.g., about 200 mg of ribavirin, with the first dose of 600 mg (or 400 mg), followed by a second dose of 400 mg (or 600 mg) at least 6 hours apart.

With respect to the Compound (1) or a pharmaceutically acceptable salt thereof plus interferon alfa plus ribavirin triple combination therapy of the present invention, the present invention contemplates and includes all combinations of the various preferred embodiments and sub-embodiments as set forth hereinabove.

For example, in one embodiment the present invention contemplates a method of treating hepatitis C viral (HCV) infection or alleviating one or more symptoms thereof in a patient that is co-infected with HIV comprising the step of administering to the patient a therapeutic combination comprising:
(a) Compound (1) or a pharmaceutically acceptable salt thereof at a dosage between about 40 mg per day and about 480 mg per day;
(b) pegylated interferon alfa-2a at a dosage of about 135 to about 180 μg/week or pegylated interferon alfa-2b at a dosage of about 0.5 μg/kg/week to about 2 μg/kg/week; and
(c) ribavirin at a dosage of between about 200 mg/day to about 1800 mg/day.

In another embodiment the present invention contemplates a method of treating hepatitis C viral (HCV) infection or alleviating one or more symptoms thereof in a patient that is co-infected with HIV comprising the step of administering to the patient a therapeutic combination comprising:
(a) Compound (1) or a pharmaceutically acceptable salt thereof at a dosage between about 120 mg per day and about 240 mg per day;
(b) pegylated interferon alfa-2a at a dosage of about 180 μg/week; and
(c) ribavirin at a dosage of between about 1000 mg/day to about 1200 mg/day.

In another embodiment the present invention contemplates a method of treating hepatitis C viral (HCV) infection or alleviating one or more symptoms thereof in a patient that is co-infected with HIV comprising the step of administering to the patient a therapeutic combination comprising:
(a) Compound (1) or a pharmaceutically acceptable salt thereof at a dosage between about 120 mg per day and about 240 mg per day;
(b) pegylated interferon alfa-2b at a dosage of about 1.5 μg/kg/week; and
(c) ribavirin at a dosage of between about 1000 mg/day to about 1200 mg/day.

Further embodiments include any of the above-mentioned embodiments, and where:
(a) the HCV infection is genotype 1 and the patient is a treatment-naïve patient; or
(b) the HCV infection is genotype 1 and the patient is a treatment-experienced patient.

Further embodiments include any of the above-mentioned embodiments, and where the Compound (1) or a pharmaceutically acceptable salt thereof is administered once a day, the interferon alpha is administered once a week and the ribavirin is administered twice a day.

With respect to the triple combination therapies of the present invention, the present invention contemplates and includes all combinations of the various preferred embodiments and sub-embodiments as set forth herein.

According to a another embodiment, the therapeutic regimen of the present invention comprises administering to a patient for at least about 4 weeks, more preferably either at least about 12 weeks or at least about 24 weeks:
(i) a therapeutically effective amount of Compound (1) or a pharmaceutically acceptable salt thereof once a day;
(ii) a therapeutically effective amount of interferon alpha once a week; and
(iii) a therapeutically effective amount of ribavirin twice a day.

An additional embodiment is directed to a packaged pharmaceutical composition comprising a packaging containing one or more doses of Compound (1) or a pharmaceutically acceptable salt thereof, an interferon alpha and ribavirin, together with written instructions directing the co-administration of Compound (1), an interferon alpha and ribavirin for the treatment of HCV infection in a patient that that is co-infected with HIV. The individual doses of Compound (1) or a pharmaceutically acceptable salt thereof, can be in the form of any of the standard pharmaceutical dosage forms, e.g. tablets, capsules, and packaged within any of the standard types of pharmaceutical packaging materials, e.g. bottles, blister-packs, etc., that may themselves be contained within an outer packaging material such as a paper/cardboard box. The written instructions will typically be provided either on the packaging material(s) itself or on a separate paper (a so-called “package insert”) that is provided together with the dosage forms within the outer packaging material. All such packaging embodiments and variations thereof are embraced by the present invention.

### Methods for Determining HIV Infection

The patient is diagnosed as having chronic HIV infection using standard diagnostic testing methods known in the art, e.g. HIV-1/HIV-2 antibody ELISA for screening, HIV Western blot for confirmation of a reactive screening test, and quantitative assay for HIV-1 or HIV-2 plasma RNA that detects HIV during the “window period” following acute infection, during which anti-HIV antibodies have not yet developed. HIV plasma RNA quantitative assays further aide in the decision regarding which specific antiretroviral therapy to start, and for monitoring the effectiveness of antiretroviral therapy.

### EXAMPLES

### I. Methods for Preparing Compound (1)

Methods for preparing amorphous Compound (1) can be found in U.S. Pat. Nos. 6,323,180, 7,514,557 and 7,585,845, which are herein incorporated by reference. Methods for preparing additional forms of Compound (1), in particular the crystalline sodium salt form, can be found in U.S. Patent Application Publication No. 2010/0093792.

### II. Formulations of Compound (1)

One example of a pharmaceutical formulation of Compound (1) include an oral solution formulation as disclosed in WO 2010/059667. Additional examples include soft-gelatin capsules containing a lipid-based liquid formulation, as disclosed in WO 2011/005646.

### III. Clinical Study

For the clinical trial described below, the Compound (1) drug product will be administered as a softgel capsule lipid-based formulation containing Compound (1) sodium salt. All references to “Compound (1)” in the below clinical study is the sodium salt form.

Clinical Study with Treatment-Naïve Patients Co-Infected with HIV

### Phase III Trial of Compound (1) in Treatment Naive (TN) and Relapser Hepatitis C Virus (HCV)—Human Immunodeficiency Virus (HIV) Coinfected Patients

### Title:

Safety and Efficacy of 120 mg and 240 mg Compound (1) Once Daily in Combination With Pegylated Interferon Alpha 2a (PegIFN) and Ribavirin (RBV) for Treatment of Chronic Hepatitis C (HCV) Genotype 1 Infection in HIV/HCV Co-infected Patients. A Multinational, Randomised, Parallel Group, Open-label Trial.

### Purpose

The aim of this trial is to evaluate the efficacy and the safety of Compound (1) given for 12 or 24 weeks in combination with PegIFN/RBV given for 24 or up to 48 weeks in HCV treatment-naive or relapsers patients coinfected with HIV. Patients who achieve reduction of HCV viral load according to Early Treatment Success (ETS) criteria will be randomized 1:1 to stop triple therapy at 24 weeks, or continue PegIFN/RBV for 48 week treatment duration. Patients without ETS continue PegIFN/RBV for 48 week treatment duration.

### Primary Outcome Measures:

Sustained Virological Response (SVR): Plasma Hepatitis C Virus (HCV) Ribonucleic Acid (RNA) level <25 IU/mL, undetected at week 12 after the planned treatment duration.

### Secondary Outcome Measures:

Virological response after 24 weeks of treatment discontinuation (SVR24): Plasma

HCV RNA level<25 IU/mL (undetected) 24 weeks after the originally planned treatment duration.
Early Treatment Success (ETS): Plasma HCV RNA level<25 IU/mL (detected or undetected) at Week 4 and HCV RNA<25 IU/mL, undetected at Week 8 and have not discontinued any study drug prior to week 24.
Alanine Aminotransferase normalisation: Alanine Aminotransferase in normal range 24 weeks after the end of the originally planned treatment duration

Arms*

Experimental: Compound (1) 12W240
patient to receive two capsules containing 120 mg Compound (1)
each once a day for 12 weeks and pegIFN/RBV for 24 or 48 weeks
Experimental: Compound (1) 24W240
patient to receive two capsules containing 120 mg Compound (1)
each once a day for 24 weeks and PegIFN/RBV for 24 or 48 weeks
Experimental Compound (1) 24 W120
Patient to receive one capsule containing 120 mg Compound (1) once
a day for 24 weeks and PegIFN/RBV for 24 or 48 weeks
*All patients will receive their first dose only as a loading dose which is twice the amount of shown maintenance dose in each treatment arm

Patient Population Criteria

Inclusion criteria (selected):
1. Chronic hepatitis C (HCV) genotype 1 infection
2. Chronic Human Immunodeficiency Virus (HIV)-1 infection
3. HCV treatment naive or HCV treatment experienced but only relapsers
4. Age 18 to 70 years
5. Antiretroviral treatment naive or on stable Highly Active Antiretroviral Therapy (HAART)
6. HCV viral load >1.000 IU/mL
7. No AIDS-defining illness during 6 months prior to screening
   Exclusion criteria (selected):
1. HCV infection of mixed genotype (1/2, 1/3, 1/4)
2. Evidence of acute or chronic liver due to chronic HCV infection
3. Hepatitis B virus (HBV) infection with presence of HBs-Ag
4. Active malignancy or history or malignancy within the last 5 years
5. Received concomitant systemic antiviral (other than antiretroviral), hematopoietic growth factor or immunomodulatory treatment in 28 days prior enrollment.
6. Decompensated liver disease, as evidenced by ascites, hepatic encephalopathy, esophageal variceal bleeding, and/or laboratory values that add up to >/=7 points according tho the Child-Turcotte-Pugh classification
7. Hemoglobin </=11 g/dL for women and </=12 g/dL for men
8. Active autoimmune disease, including autoimmune hepatitis
9. Known hypersensitivity to any ingredient of the study drugs

### Interim Clinical Results and Analysis

### Background:

Compound (1) (faldaprevir or FDV) is a HCV NS3/4A protease inhibitor (PI) in late stage clinical development for the treatment of HCV genotype 1 (GT1) infection. In this open-label, sponsor-blinded, Phase III trial, the efficacy and safety of FDV plus pegylated interferon alfa-2a and ribavirin (PegIFN/RBV) was assessed in individuals coinfected with HCV and HIV.

### Methods:

Individuals co-infected with HCV/HIV who were HCV treatment-naïve (TN) or relapsed after previous HCV therapy were randomized to receive FDV 120 or 240 mg QD for 12 or 24 weeks, in combination with PegIFN (180 μg once weekly) and weight-based RBV for 24 or 48 weeks (response-guided). Patients on efavirenz- or PI-based antiretroviral therapy (ART) were allocated to receive FDV 240 mg or 120 mg QD, respectively; those receiving other ART or no ART were randomized to either FDV dose. Interim data remains blinded for FDV dose. Week 12 interim data are presented.

### Results:

308 patients were randomized and treated: 96% were receiving ART; for HCV therapy, 78% were TN and 22% were relapsers; 81% were male; 78% were caucasian; 4% had liver fibrosis equivalent to F4 and 13% had Fibroscan >13 kPa; 80% had baseline HCV RNA ≧800,000 IU/mL. 77% had GT1a. Virologic responses are shown (Table).

| | | | |
|---|---|---|---|
| Virologic response, | TN | Relapsers | Total |
| n (%) | (N = 239) | (N = 69) | (N = 308) |

| | | | | |
|---|---|---|---|---|
| Week 4 | BLQ | 191 (80)^{a} | 63 (91)^{b} | 254 (82) |
| | BLD | 143 (60) | 51 (74) | 194 (63) |
| Week 12 | BLQ | 206 (86)^{c} | 64 (93)^{d} | 270 (88) |
| | BLD | 195 (82) | 63 (91) | 258 (84) |

| | | | |
|---|---|---|---|
| ETS (Week 4 BLQ | 184 (77)^{e} | 61 (88) | 245 (80) |
| and Week 8 BLD) | | | |

^{a}Missing n = 6;
^{b}Missing n = 1;
^{c}Missing n = 9;
^{d}Missing n = 3;
^{e}Missing n = 4;
BLQ, HCV RNA below limit of quantification (<25 IU/mL, detected);
BLD, HCV RNA below limit of detection (<15 IU/mL, not detected), by COBAS ®TaqMan ® v2.0 (Roche);
ETS, early treatment success

HCV RNA was BLD at Week 4/Week 12 in 64%/64% of patients not on ART, and in 63%/85% of patients receiving ART. The preliminary, on-treatment efficacy for this treatment regimen in HCV/HIV co-infected patients is quite good and comparable to that seen in HCV monoinfected patients. For example, in a prior study in HCV GT1 mono-infected TN patients treated with 240 mg QD FDV plus PegIFN/RBV (Boehringer Ingelheim, data on file, Clinical Trial Report for study 1220.5, U12-2567-01), HCV RNA was BLQ at week 4 and BLD at week 12 in 94% (133/142) and 93% (132/142) of patients, respectively. In comparison, the interim data presented above shows BLQ at week 4 and BLD at week 12 in 80% and 82% of the HCV/HIV co-infected TN patient population. These preliminary on-treatment efficacy data are promising given that a) 17% of the coinfected patients had cirrhosis (patients with cirrhosis were not included in the 1220.5 study), b) preliminary efficacy data in HCV/HIV coinfected represent sponsor-blinded results of both FDV dose groups (e.g., 120 mg QD and 240 mg QD FDV), and c) the HCV GT1/HIV co-infected patient population is historically a more difficult-to-treat patient population with substantially lower HCV cure rates with the current standard of care treatment of pegIFN and RBV when compared to HCV GT1 monoinfected patients. The most frequent adverse events (AEs) were those known for PegIFN/RBV treatment: nausea (37%), fatigue (33%) and diarrhea (27%). Serious AEs were reported in 32 (10%) patients (including 2 deaths; none considered related to study medications). To date, 18 patients discontinued early due to AEs. No patient on ART experienced loss of HIV RNA suppression.

### CONCLUSIONS

In this interim analysis, FDV plus PegIFN/RBV provided high early virologic response rates in HCV GT1 patients coinfected with HIV at Weeks 4 and 12. The efficacy and safety profile was comparable to that observed in HCV mono-infected TN patients treated with FDV and pegIFN/RBV.

1. A method of treating hepatitis C viral (HCV) infection or alleviating one or more symptoms thereof in a patient that is co-infected with the Human Immunodeficiency Virus (HIV) comprising the step of administering to the patient a therapeutic combination comprising:
(a) a compound of the following formula (1) or a pharmaceutically acceptable salt thereof: 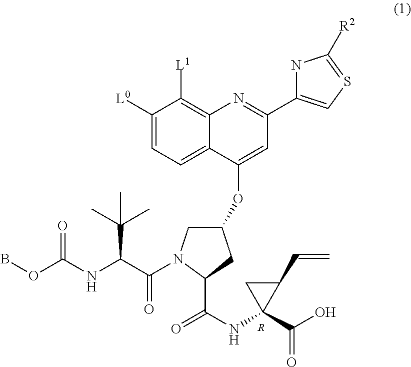
wherein B is 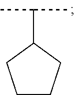
L⁰ is MeO—; L¹ is Br; and R² is 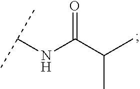
(b) interferon alpha; and
(c) ribavirin.

2. The method according to claim 1, wherein the patient has HCV subtype 1.

3. The method according to claim 1, wherein the patient has HCV subtype 1a.

4. The method according to claim 1, wherein the patient has an HIV-1 infection.

5. The method according to claim 1, wherein the patient is already on anti-retroviral therapy.

6. The method according to claim 1, wherein said patient is a treatment-naive patient.

7. The method according to claim 1, wherein said patient is a treatment experienced patient.

8. The method according to claim 1, wherein the HCV-RNA levels of said patient are reduced to a less than detectable level as a result of the treatment.

9. The method according to claim 1, wherein said therapeutic combination is administered for at least 4 weeks.

10. The method according to claim 1, wherein said therapeutic combination is administered for at least 12 weeks.

11. The method according to claim 1, wherein said therapeutic combination is administered for at least 24 weeks.

12. The method according to claim 1, wherein compound (1) or a pharmaceutically acceptable salt thereof is administered at a maintenance dosage of at least 40 mg per day.

13. The method according to claim 1, wherein compound (1) or a pharmaceutically acceptable salt thereof is administered at a maintenance dosage between about 40 mg per day and about 480 mg per day.

14. The method according to claim 1, wherein compound (1) or a pharmaceutically acceptable salt thereof is administered at a maintenance dosage between about 120 mg per day and about 240 mg per day.

15. The method according to claim 1, wherein compound (1) is administered in the form of its sodium salt.

16. The method according to claim 1, wherein said ribavirin is administered at a dosage between about 200 mg/day and about 1800 mg/day.

17. The method according to claim 1, wherein said ribavirin is administered at a dosage between about 800 mg/day and about 1200 mg/day.

18. The method according to claim 1, wherein said interferon alpha is administered once a week.

19. The method according to claim 1, wherein said interferon alpha is a pegylated interferon alfa.

20. The method according to claim 1, wherein said pegylated interferon alfa is pegylated interferon alfa-2a or pegylated interferon alfa-2b.
